(19) United States
(12) Patent Application Publication
(10) Pub. No.: US 2025/0297547 A1
(43) Pub. Date: Sep. 25, 2025

(54) ROCK TYPE IDENTIFICATION FOR DRILLING OPERATIONS

(71) Applicant: Saudi Arabian Oil Company, Dhahran (SA)

(72) Inventors: Osama Alsalmi, Khobar (SA); Andrew Rees, Dhahran (SA); Fahd A. Almalki, Dhahran (SA); Francis Laurent Elisabeth, Al Khobar (SA); Maria Elena Bentosa Gutierrez, Dhahran (SA)

(21) Appl. No.: 18/612,524

(22) Filed: Mar. 21, 2024

(51) Int. Cl. E21B 49/00; E21B 44/00; E21B 45/00; G01N 33/24; G01V 20/00; G06N 20/00

(52) U.S. Cl. CPC E21B 49/00; E21B 44/00; G01N 33/24; G01V 20/00; G06N 20/00; E21B 45/00; E21B 2200/20

(57) ABSTRACT

Systems and methods include obtaining well log data and core sample data of a subsurface formation; generating, based on the well log data and the core sample data, an unconfined compressive strength log for the subsurface formation; using an unsupervised machine learning model to form rock type clusters based on the unconfined compressive strength log and the well log data; forming a training dataset including the well log data, the training dataset labeled based on the rock type clusters; training a supervised machine learning model using the training dataset. While drilling a well in the subsurface formation, logging-while-drilling data is obtained from drilling equipment used to drill the well; and rock types in the subsurface formation are determined using the supervised machine learning model and the logging-while-drilling data.

### TECHNICAL FIELD

The present disclosure relates to methods and systems for rock type identification for drilling operations.

### BACKGROUND

Geosteering is the process of adjusting the well trajectory of a wellbore while drilling the well to stay within a specific geological target. Geosteering can be important during the exploration and exploitation of hydrocarbons (e.g., oil and gas) from subsurface formations. For example, when drilling horizontal wells, geosteering enables the direction of the well to be controlled to stay within the target zone of the subsurface formation (e.g., the sweet spot) to improve the well productivity. Geologists can use well logs and/or data from nearby wells to infer rock properties of the subsurface formation to make decisions on the direction of the well and other drilling parameters.

### SUMMARY

Rocks in conventional reservoirs can have porosity and permeability broken down into facies and rock types that have unique reservoir quality descriptors. The reservoir quality descriptors can be readily tied to specific conventional log properties such as gamma ray, density and sonic. Unconventional reservoirs can have significantly reduced porosity and permeability compared with conventional reservoirs that narrows the band for facies analysis and rock typing increasing the difficulty of identifying the facies and rock types. Subtle changes that are not easily discernible and/or low resolution logs result in difficulty discriminating different rock types and an inability to produce electro-facies that can be calibrated to cores to determine rock types.

This disclosure describes systems and methods for determining rock types for a subsurface formation. These systems and methods are useful for determining rock types in unconventional (e.g., tight, low porosity, low permeability) reservoirs in the subsurface formation. A data processing system (e.g., a computing system or a control system) can obtain well log and core sample data of a subsurface formation. Based on the well log data and the core sample data, the data processing system can generate an unconfined compressive strength log for the subsurface formation. The data processing system can use an unsupervised machine learning model to form rock type clusters based on the unconfined compressive strength log and the well log data. The data processing system can form a training set including the well log data where the training dataset can be labeled based on the rock type clusters. The data processing system can train a supervised machine learning model using the training dataset. While drilling a well in the subsurface formation, the data processing system can obtain logging-while-drilling data from drilling equipment used to drill the well, and the data processing system can determine rock types in the subsurface formation using the supervised machine learning model and the logging-while-drilling data.

Implementations of the systems and methods of this disclosure can provide various technical benefits. The data processing system can determine rock types while drilling a well where the rock types are calibrated to rock types from core samples taken from the subsurface formation. Determining the rock types while drilling enables adjustment of drilling parameters in real-time (e.g., as the well is being drilled) to, for example, geosteer the well to stay within desirable rock types for hydrocarbon production. Other drilling parameters such as rate of penetration or weight on bit can also be adjusted based on the determined rock types. Three-dimensional static and dynamic geological models can be updated in real-time based on the determined rock types. Complications during drilling and/or hydraulic fracturing can be reduced based on the determined rock types and rock properties related to the rock type as compared to drilling or hydraulic fracturing in unknown rock types. Rock types can act as a proxy for hydrocarbon storage potential since higher porosity can lead to higher storage capacity. Geosteering wells based on the identified rock types can increase the productivity of a well by steering the well to stay within a rock type with a higher porosity.

The details of one or more embodiments of these systems and methods are set forth in the accompanying drawings and the description below. Other features, objects, and advantages of these systems and methods will be apparent from the description and drawings, and from the claims.

### BRIEF DESCRIPTION OF THE DRAWINGS

FIG. 1 is a schematic illustrating a wireline operation.

FIG. 2 is a flow chart of an example method for determining rock types.

FIG. 3 is a flow chart of an example method for determining unconfined compressive strength (UCS) of a subsurface formation.

FIG. 4 is a flow chart of an example method for obtaining and real-time logging-while-drilling data.

FIG. 5 shows example images of rock from conventional and unconventional reservoirs.

FIG. 6 shows depositional facies and rock types from an example subsurface formation.

FIG. 7 is an example plot of permeability versus porosity for rock from the example subsurface formation.

FIG. 8 another example plot of permeability versus porosity for rock from the example subsurface formation.

FIG. 9 is an example plot of reservoir porosity type as determined by thin section analysis.

FIG. 10 is an example composite plot of porosity and cement type from the example subsurface formation.

FIG. 11 is an example workflow for generating an unconfined compressive strength (UCS) log.

FIG. 12 is an example cross plot of core UCS from micro-rebound hammer measurements and core porosity.

FIG. 13 is an example cross plot of log UCS and core porosity.

FIG. 14 is a composite plot of conventional wireline logs with core sample data and UCS data.

FIG. 15A illustrates example geological characteristics of rock type 1.

FIG. 15B illustrates example geological characteristics of rock type 2.

FIG. 16 is an example cross plot of log UCS versus core porosity using data from FIG. 14.

FIG. 17 is an example cross plot of rate of penetration versus UCS from three wells in the example subsurface formation.

FIG. 18 is a schematic showing example input parameters to an artificial intelligence model to predict drilling parameters.

FIG. 19 is an example plot of clusters formed by an unsupervised machine learning model.

FIG. 20 is an example elbow plot of determining the number of clusters to be used by an unsupervised machine learning model.

FIG. 21 is an example bar chart of the distribution of rock types within example training data.

FIG. 22 is an example bar chart of an equalized distribution of rock types of the example training data using random under sampling.

FIG. 23 is an example accuracy plot showing performance of a variety of supervised machine learning classifiers.

FIG. 24 is an example confusion matrix for an extra trees classifier.

FIG. 25 is an example feature importance plot for the extra trees classifier of FIG. 24.

FIG. 26 illustrates hydrocarbon production operations that include field operations and computational operations, according to some implementations.

FIG. 27 is a block diagram illustrating an example computer system used to provide computational functionalities associated with described algorithms, methods, functions, processes, flows, and procedures according to some implementations of the present disclosure.

Like reference symbols in the various drawings indicate like elements.

### DETAILED DESCRIPTION

This disclosure describes systems and methods for determining rock types for a subsurface formation. A data processing system (e.g., a computing system or a control system) can obtain well log and core sample data of a subsurface formation. Based on the well log data and the core sample data, the data processing system can generate an unconfined compressive strength log for the subsurface formation. The data processing system can use an unsupervised machine learning model to form rock type clusters based on the unconfined compressive strength log and the well log data. The data processing system can form a training set including the well log data where the training dataset can be labeled based on the rock type clusters. The data processing system can train a supervised machine learning model using the training dataset. While drilling a well in the subsurface formation, the data processing system can obtain logging-while-drilling data from drilling equipment used to drill the well, and the data processing system can determine rock types in the subsurface formation using the supervised machine learning model and the logging-while-drilling data.

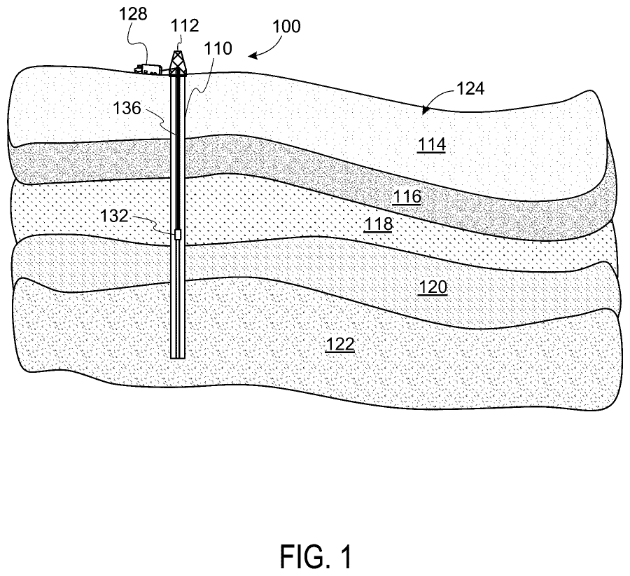

FIG. 1 illustrates a wireline operation 100 (e.g., a well logging operation) in which a wellbore 110 extends downhole from a wellhead 112. The wellbore 110 is a vertical wellbore but wireline operations can also be performed in other wellbores, for example, slanted or horizontal wellbores. In the wireline operation 100, the wellbore 110 penetrates through five layers 114, 116, 118, 120, 122 of a subsurface formation 124. A control truck 128 lowers a logging tool 132 (e.g., a sidewall coring tool) down the wellbore 110 on a wireline 136.

The logging tool 132 is a string of one or more instruments with sensors operable to measure petrophysical properties of the subsurface formation 124. For example, logging tools can include resistivity logs, borehole image logs, porosity logs, density logs, or sonic logs. Resistivity logs measure the subsurface electrical resistivity, which is the ability to impede the flow of electric current. These logs can help differentiate between formations filled with salty waters (good conductors of electricity) and those filled with hydrocarbons (poor conductors of electricity). Porosity logs measure the fraction or percentage of pore volume in a volume of rock using acoustic or nuclear technology. Acoustic logs measure characteristics of sound waves propagated through the well-bore environment. Nuclear logs utilize nuclear reactions that take place in the downhole logging instrument or in the formation. Density logs measure the bulk density of a formation by bombarding it with a radioactive source and measuring the resulting gamma ray count after the effects of Compton scattering and photoelectric absorption. Sonic logs provide a formation interval transit time, which is typically a function of lithology and rock texture but particularly porosity. The logging tool includes a piezoelectric transmitter and receiver and the time taken for the sound wave to travel the fixed distance between the two is recorded as an interval transit time.

As the logging tool 132 travels downhole, measurements of formation properties are recorded to generate a well log. In the illustrated operation, the data are recorded at the control truck 128 in real-time. Real-time data are recorded directly against measured cable depth. In some well-logging operations, the data is recorded at the logging tool 132 and downloaded later. In this approach, the downhole data and depth data are both recorded against time. The two data sets are then merged using the common time base to create an instrument response versus depth log.

In the wireline operation 100, the well logging is performed on a wellbore 110 that has already been drilled. In some operations, well logging is performed in the form of logging while drilling techniques. In these techniques, the sensors are integrated into the drill string and the measurements are made in real-time, during drilling rather than using sensors lowered into a well after drilling.

Using a wireline coring tool, core samples can be obtained in addition to obtaining well logs. A core sample is a usually cylindrical piece of the subsurface formation that is removed by a special drill and brought to the surface. Core samples can be used to measure petrophysical properties of the subsurface formation such as grain size, porosity, and permeability. Core samples can also be used to measure geomechanical properties of the subsurface formation such as Young's modulus, Poisson's ratio, and shear modulus. Core samples can be taken from the sidewalls of a drilled well. When sidewall core samples are repeated along the length of the well, the properties measured from the core samples can be compared and correlated with well logging measurements.

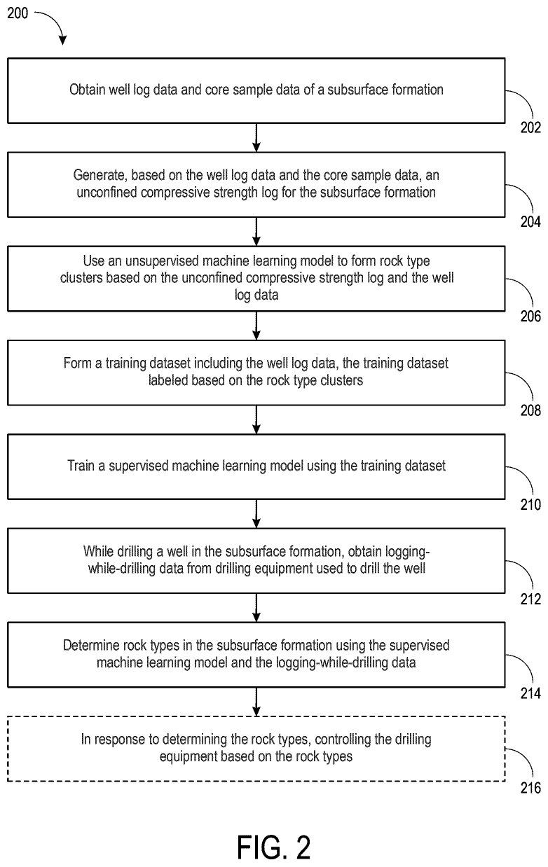

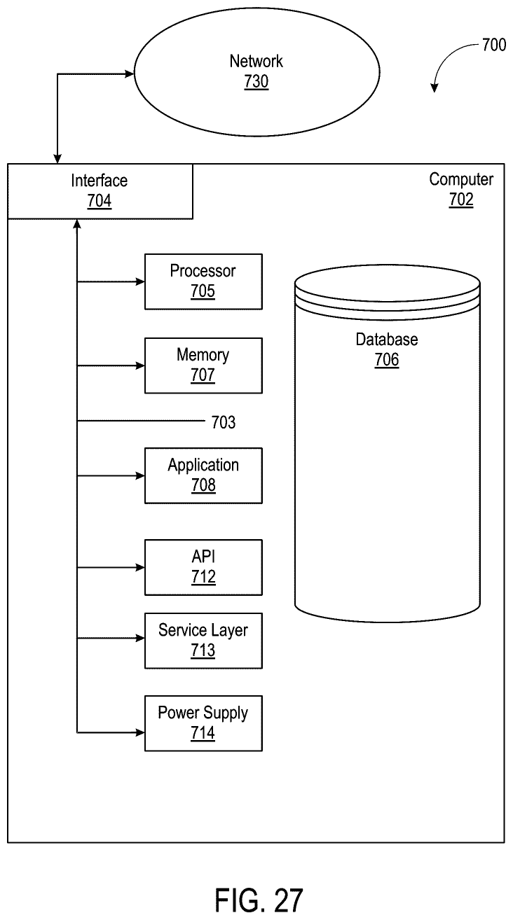

FIG. 2 is a flow chart of an example method 200 for determining rock types for a subsurface formation. The method 200 can be implemented on a data processing system such as a computer or control system (e.g., the computer system of FIG. 27). The method 200 can be used to determine rock types in real-time while drilling a well in the subsurface formation.

At step 202, the data processing system obtains well log data and core sample data of the subsurface formation. The well log data includes, for example, one or more of gamma ray (GR) logs, density (RHOB) logs, total porosity (PHIT) logs, sonic (DT) logs, rate of penetration (ROP) logs, torque logs, revolutions per minute (RPM) logs, hook loads, flow rates, standpipe pressure (SPP), and mechanical specific energy (MSE). Core sample data can include one or more of core facies data, UCS data, micro-rebound UCS, porosity, permeability, x-ray diffraction, and thin sections. Other types of well log data and/or core sample data are also possible.

At step 204, the data processing system generates a UCS log for the subsurface formation based on the well log data and the core sample data. For example, the data processing system correlates laboratory measurements from core samples with the well log data to generate the UCS log.

At step 206, the data processing system uses an unsupervised machine learning model to form rock type clusters based on the UCS log and the well log data. Unsupervised machine learning models can determine clusters for multi-dimensional data that may not be easily inferred by other methods. For example, the data processing system uses a k-means clustering machine learning model to identify rock type clusters. The k-means clustering model can take as input the UCS log, a density log, a gamma ray log and a porosity log and output cluster centers for a specified number of rock type clusters (e.g., 4 rock type clusters). In some implementations, the data processing system determines the number of clusters based on an elbow plot.

In some implementations, the data processing system reduces the number of inputs to the unsupervised machine learning model using a principal component analysis (PCA). A PCA combines input features to form principal components that describe the input data in fewer variables. For example, the data processing system can reduce the UCS log, the density log, the gamma ray log, and the porosity log to two principal components thereby reducing the input dimensions from four dimensions (e.g., four logs) to two dimensions (e.g., the two principal components).

At step 208, the data processing system forms a training dataset including the well log data. The data processing system labels the training dataset based on the rock type clusters. For example, the data processing system labels the well log data according to the rock type determined by the unsupervised machine learning model. The data processing system can verify the clusters based on core sample data. The training dataset can include well log data that can be obtained from logging-while-drilling equipment such as ROP logs, gamma ray logs, weight on bit logs, and MSE logs. The data processing system can label the training dataset based on the data clustered by the unsupervised machine learning model. Each data point in the well log data can be indexed by depth.

At step 210, the data processing system trains a supervised machine learning model using the training dataset. The supervised machine learning model can be a classifier model. For example, an extra trees classifier, an extreme gradient boosting classifier, a random forest classifier, a gradient boosting classifier, a k-neighbors classifier, a decision tree classifier, a quadratic discriminant classifier, a naive Bayes classifier, a logistic regression classifier, a linear discriminant classifier, a ridge classifier, an Ada boost classifier, or a support vector machine classifier. The supervised machine learning model takes as input well log data (e.g., drilling parameters, logging-while-drilling data) and predicts rock types of the subsurface formation as output.

At step 212, while drilling a well, the data processing system obtains logging-while-drilling data from drilling equipment being used to drill the well. The logging-while-drilling data can include ROP logs, gamma ray logs, weight on bit logs, and MSE logs. The data processing system can obtain the logging-while-drilling data in real-time.

At step 214, the data processing system determines the rock types of the subsurface formation using the supervised machine learning model and the logging-while-drilling data. For example, the data processing system inputs the logging-while-drilling data into the trained supervised machine learning model which produces a predicted rock type corresponding to the input data. The data processing system can determine the rock type in real-time (e.g., while the well is being drilled).

Real-time or near real-time processing refers to a scenario in which received data (e.g., logging-while-drilling data) are processed as made available to systems and devices requesting those data immediately (e.g., within milliseconds, tens of milliseconds, or hundreds of milliseconds) after the processing of those data are completed, without introducing data persistence or store-then-forward actions. In this context, a real-time data processing system is configured to process an emergency alert message as it arrives and broadcast the emergency alert message as quickly as possible (though processing latency may occur). Though data can be buffered between module interfaces in a pipelined architecture, each individual module operates on the most recent data available to it. The overall result is a workflow that, in a real-time context, receives a data stream (e.g., logging-while-drilling data) and outputs processed data (e.g., determined rock types) based on that data stream in a first-in, first out manner. However, non-real-time contexts are also possible, in which data are stored (either in memory or persistently) for processing at a later time. In this context, modules of the data processing system do not necessarily operate on the most recent data available.

At step 216, the data processing system can control drilling equipment based on the determined rock types. In some implementations, the data processing system steers the drilling equipment based on the determined rock type. For example, the data processing system steers the drilling equipment to increase contact time with desirable rock types. When the determined rock type is a desirable reservoir rock type, the data processing system can steer the drilling equipment to stay within the desirable reservoir zone. Alternately, or additionally, when the determined rock type changes from a desirable rock type to an undesirable (or less desirable) rock type (or vice versa), the data processing system steers the drilling equipment in the direction of the desirable rock type. Steering drilling equipment to place a well in the subsurface formation based on the determined rock types can provide better performance than steering the drilling equipment, for example, based on gas reads. The rock types can be determined independent of drilling dynamics, such as shocks and vibrations, that can affect the gas response even when drilling in a desired rock type.

In some implementations, the data processing system controls the rate of penetration of the drilling equipment, the weight on bit, and/or the torque of the drilling equipment. For example, if the determined rock type is a softer rock, the data processing system can implement a relatively higher ROP. Each rock type can correspond with a range of ROP values when other drilling parameters remain constant. The data processing system can include other drilling parameters in the control of the drilling equipment (e.g., drilling fluid properties, drill bit design, drilling techniques, etc.).

In some implementations, the data processing system updates a geological model (e.g., a three dimensional static and/or dynamic reservoir model) based on the determined rock types. The data processing system can update the geological model in real time enabling operators to have up-to-date information for decision making.

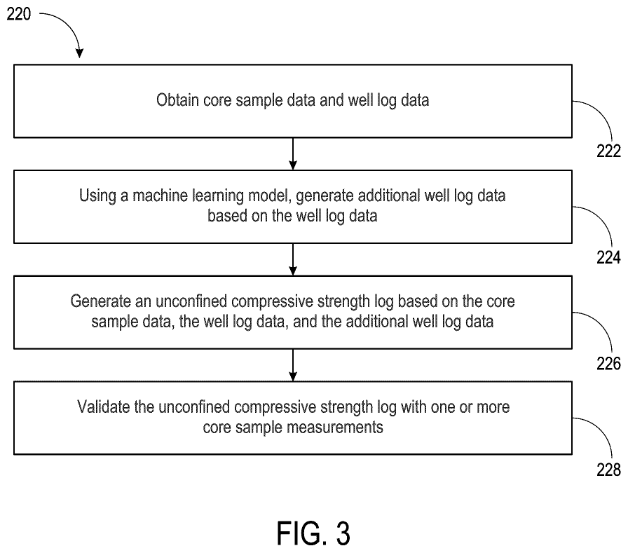

FIG. 3 is a flow chart of an example method 220 for generating a UCS log for a subsurface formation. For example, step 204 of method 200 can incorporate some or all of the steps of method 220 for generating a UCS log. The method 220 can be implemented on a data processing system such as a computer or control system (e.g., the computer system of FIG. 27).

At step 222, the data processing system obtains well log and core sample data. The well log data can include gamma ray logs, hook load logs, RPM logs, ROP logs, torque logs, WOB logs, flow rate logs, SPP logs, etc. The well log data can be sanitized to remove factors affecting rock response not related to actively drilling the well.

At step 224, using a machine learning model, the data processing system generates additional well log data based on the obtained well log data. For example, the data processing system generates compressional slowness (DTCO) logs, shear slowness (DTSM) logs, and bulk density (RHOB) logs. Examples of machine learning models that can be used to generate the additional log data include random forest, XGBoost, and convolutional neural networks. The data processing system can select a machine learning model based on a performance metric of the machine learning model. For example, the data processing system can select a machine learning model that has a lowest root square mean error (RMSE) when the trained model is tested on a validation dataset. The machine learning model can capture geologic patterns at different scales by convolving the input to the machine learning model with different sized filters corresponding to the different scales. In some implementations, the order in which the additional log data is generated can affect the quality of the additional log data. For example, the additional log data can be generated starting with ROP, then WOB, torque, and gamma ray.

At step 226, the data processing system generates a UCS log based on the core sample data, the well log data, and the additional well log data. For example, the data processing system can generate a UCS log based on the DTCO, DTSM and RHOB logs.

At step 228, the data processing system validates the UCS log with one or more core sample measurements. For example, the data processing system uses one or more of micro-rebound hammer data, x-ray diffraction data, thin section data, core UCS data, core porosity data, and core permeability data to validate the generated UCS log. The data processing system uses the core sample measurements as a quality control for the generated UCS log. When the generated UCS log matches well with the core sample measurements (e.g., within a specified envelope surrounding the core sample measurements, such as within 5% or 10% of the measured value), the generated UCS log is determined to be reliable.

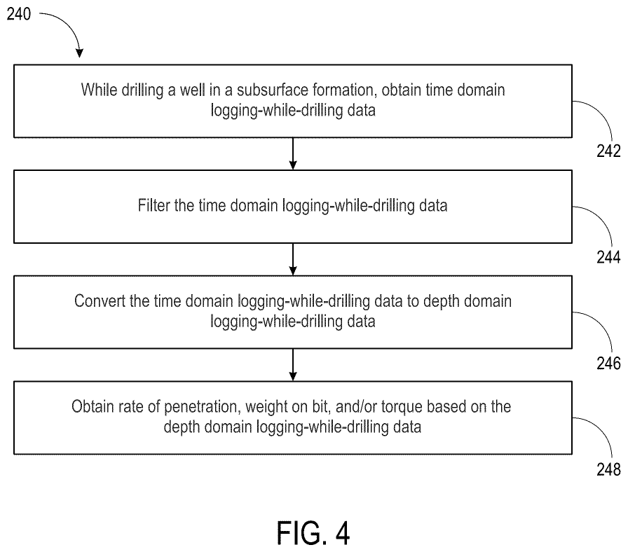

FIG. 4 is flow chart of an example method 240 for generating depth domain logging-while-drilling-data. The method 240 can be implemented on a data processing system such as a computer or control system (e.g., the computer system of FIG. 27). The data generated by method 240 can be used to obtain the logging-while-drilling data for input to the supervised machine learning model of method 200.

At step 242, the data processing system obtains time domain logging-while-drilling data. The logging-while-drilling-data can include drilling parameters (e.g., ROP, WOB, SPP, torque). The logging-while-drilling data can also include well log data (e.g., gamma ray, density, porosity logs) acquired while drilling the well.

At step 244. the data processing system filters the time domain logging-while-drilling data. For example, the data processing system filters the time domain logging-while-drilling data to remove effects arising from operational issues such as torque spikes, drill bit wear, tool failures, etc. The data processing system filters the time domain data to capture the portions of the data that can be directly correlated to the rock properties under consistent drilling conditions.

At step 246, the data processing system converts the time domain logging-while-drilling data to depth domain logging-while-drilling data. The data processing system filters the time domain data to remove data corresponding to operational activities over time that do not increase the depth of the well to form the depth domain data.

At step 248, the data processing system obtains ROP, WOB, and/or torque logs based on the depth domain logging-while-drilling data. The obtained data can be used to predict DTCO, DTSM, and RHOB logs for rock strength calculation and determining rock types of the subsurface formation.

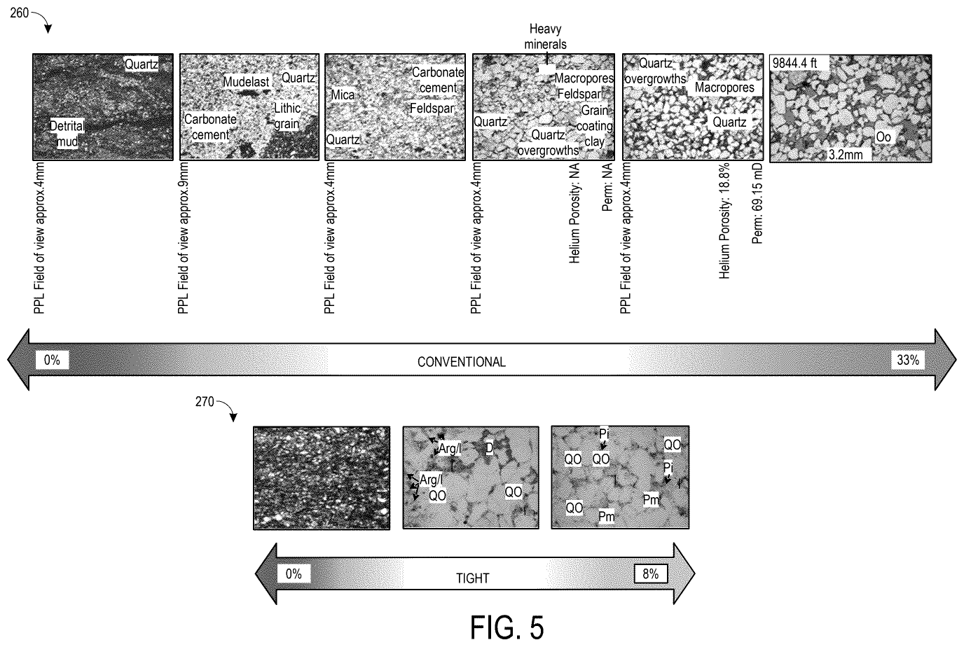

FIG. 5 shows example images of rock from conventional reservoirs 260 and unconventional reservoirs 270 (e.g., tight reservoirs). Rocks with conventional porosity and permeability can be easily broken down into facies and rock types that have unique reservoir quality descriptors and can be readily tied to specific conventional log properties such as Gamma Ray, Density and Sonic. In the conventional reservoirs 260, porosity of the rock ranges from 0% porosity on the left end of the scale to 33% at the right end of the scale. As the porosity increases, larger grains and pores are visible in the images. Unconventional reservoirs have significantly reduced porosity and permeability that narrows the band for facies analysis and rock typing. In the unconventional reservoirs 270, the porosity ranges from 0% on the left end of the scale to 8% on the right end of the scale. The subtle changes in the rocks in the unconventional reservoirs 270 can result in an inability to determine rock types or faces of the unconventional reservoir.

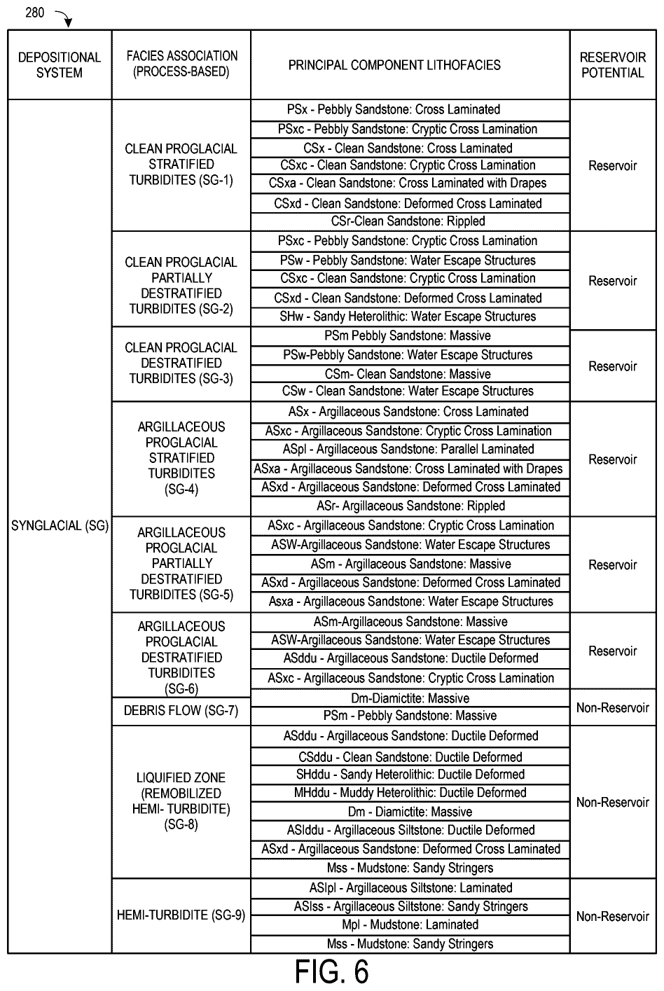

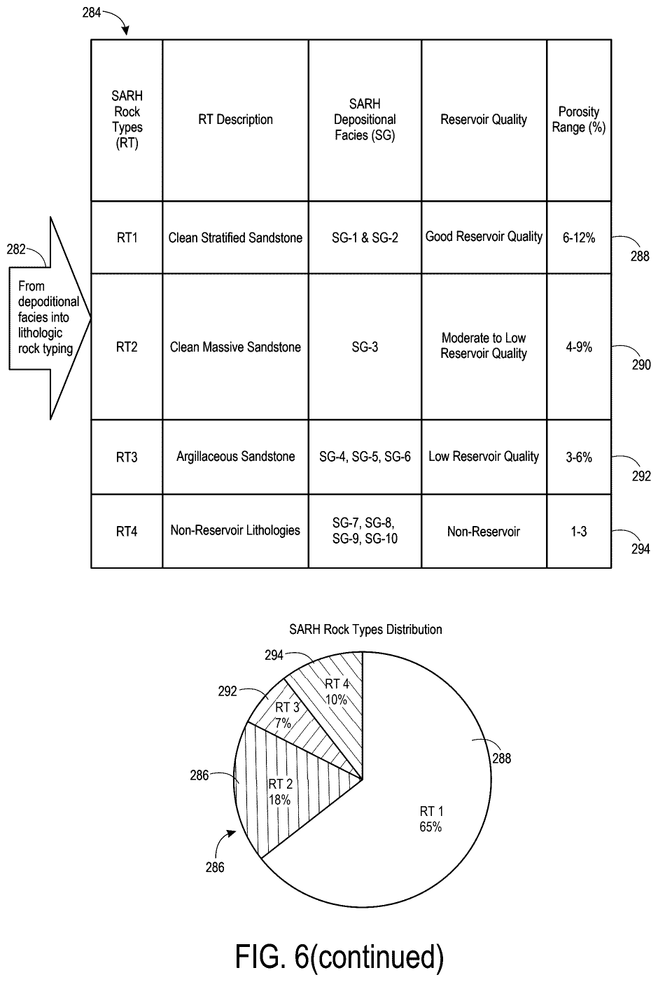

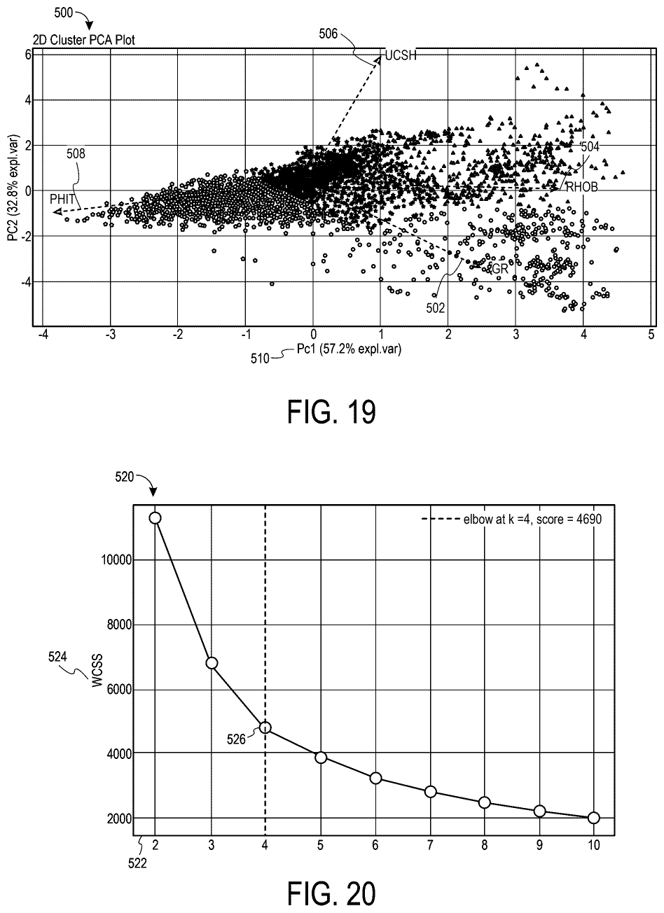

FIGS. 6-25 show data from an example implementation of methods 200, 220, and 240. The data in the example implementation presented is taken from a hydrocarbon appraisal field (field A) which can be considered “unconventional” in nature.

FIG. 6 shows depositional facies 280 from field A combined 282 into four rock types 284. Pie chart 286 shows the distribution of rock types within field A. Rock type 1 (RT1) 288 includes clean stratified sandstone. RT1 288 indicates good reservoir quality with porosity in the range of 6-12%. RT1 288 forms 65% of field A. Rock type 2 (RT2) 290 includes clean massive sandstone with moderate to low reservoir quality. The porosity of RT2 290 is in the range of 4-9% and RT2 forms 18% of field A. Rock type 3 (RT3) 292 includes argillaceous sandstone with low reservoir quality having a porosity in the range of 3-6%. RT3 292 forms 7% of field A. Rock type 4 (RT4) 294 includes non-reservoir lithologies having porosity in the range of 1-3%. RT4 294 forms 10% of field A.

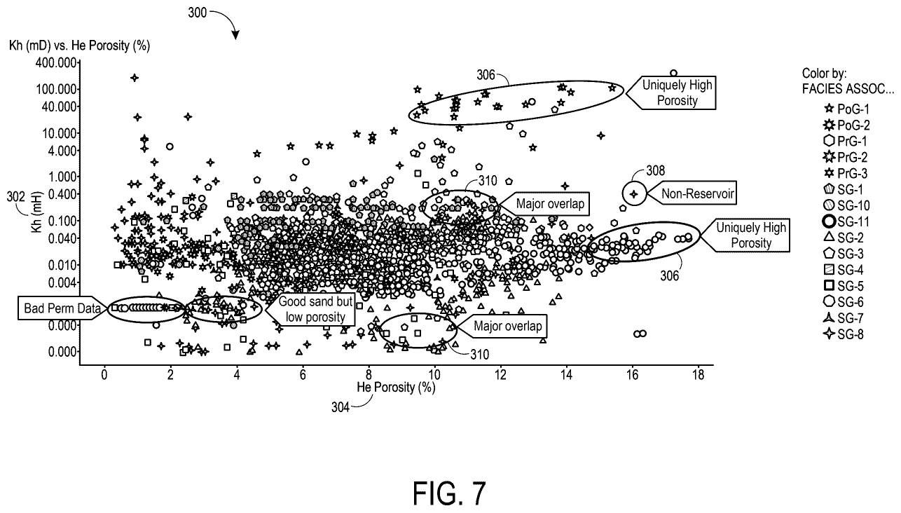

FIG. 7 is a plot 300 of permeability 302 versus porosity 304 of rock in field A. In many unconventional reservoirs such as field A, no relationship exists between core derived depositional facies and reservoir quality (porosity and permeability). For example, there are regions of uniquely high permeability data 306 interspersed with non-reservoir data 308. Additionally, there are regions of major overlap 310 between different rock types.

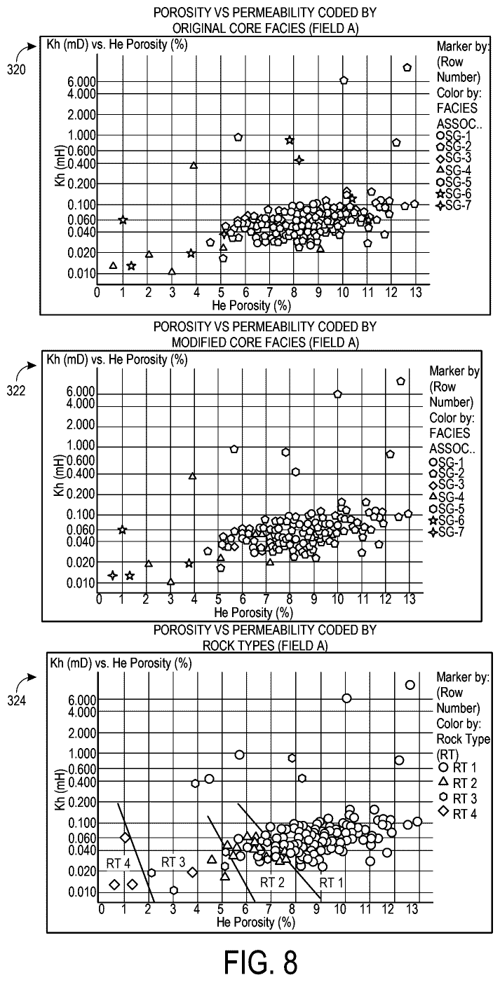

FIG. 8 shows plots of porosity versus permeability. Plot 320 is coded according to original core facies. Plot 322 is coded according to modified core facies. Plot 324 is coded according to rock types. There is no discernable relationship between porosity, permeability, and original core facies (as shown in plot 320) or modified core facies (as shown in plot 322). Neither original core facies nor modified core facies can be used to predict reservoir quality in these circumstances. On the other hand, a good segregation of data points exists when porosity and permeability is coded by rock types (shown in plot 324). Rock types have a discrete reservoir quality range and can be used in predictive geological workflows.

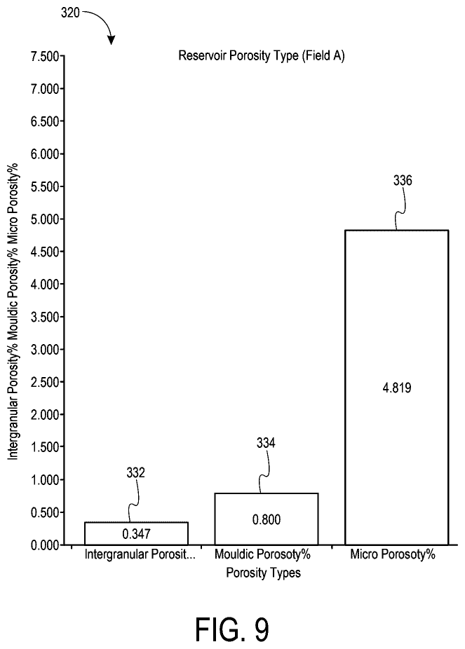

FIG. 9 is a plot 330 of reservoir porosity type as determined by thin section analysis. Intergranular porosity 332 forms 0.347% porosity. Moldic porosity 334 form 0.800% porosity. Micro porosity 336 forms 4.819% porosity. The porosity systems in Field A wells are dominated by micro-porosity held within illite clay fibers. This is unusual for reservoir sands where porosity is intergranular or secondary in origin. The presence of clays to derive porosity leads to a softer and/or weaker rock (e.g., lower UCS-Rock Type 1).

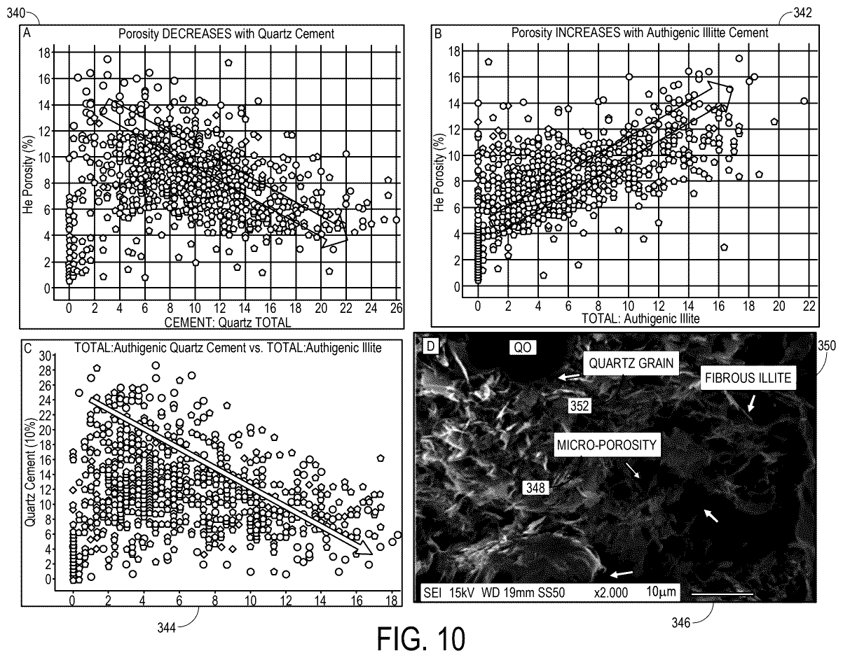

FIG. 10 is a composite plot of porosity and cement type (e.g., quartz, illite clay) for field A. In plot 340, porosity is plotted versus quartz cement. As the quartz cement increases, the porosity decreases resulting in a harder rock and higher UCS. Plot 342 shows porosity versus authigenic illite where porosity increases with an increase in soft illite producing rock types with low UCS. The inverse correlation between cement types demonstrated in plot 344 suggests earlier illite cement inhibited the nucleation of quartz to the grain protecting rock porosity. Micrograph 346 shows microporosity 348, fibrous illite 350 and quartz grain 352 at 2,000× zoom using a scanning electron microscope. Micrograph 346 shows that the microporosity 348 is held within the fibrous illite 350.

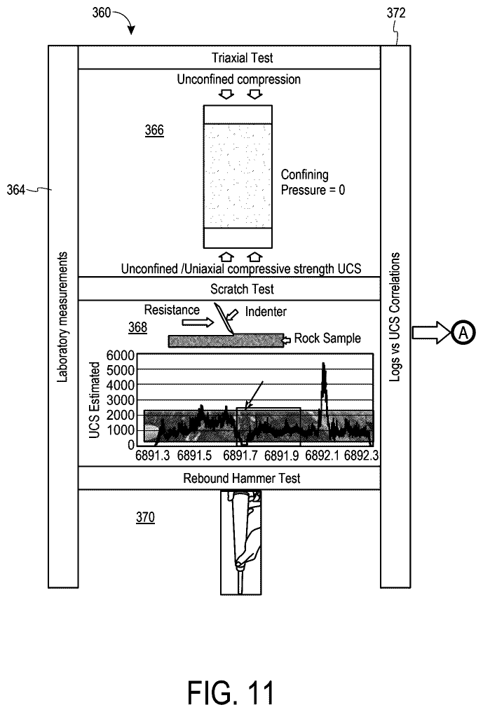

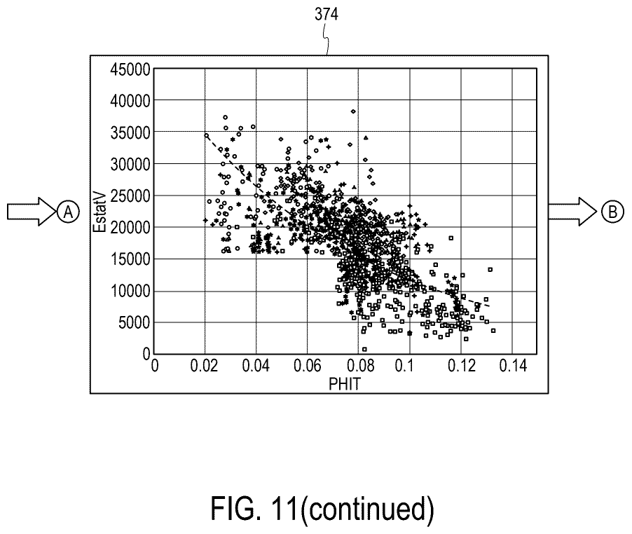

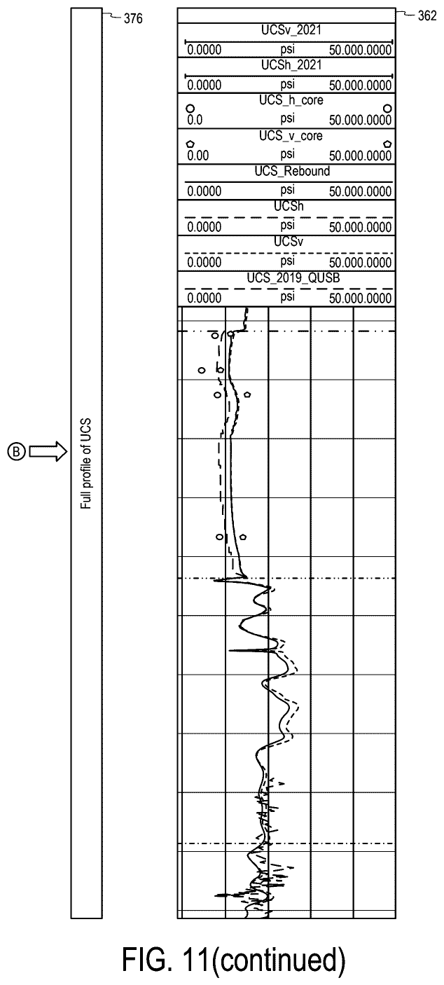

FIG. 11 is an example workflow 360 for generating a UCS log 362. Laboratory measurements 364 including triaxial test 366, scratch test 368, and micro rebound hammer test 370 are used to measure UCS from core samples acquired from the subsurface formation. The laboratory measurements 364 are correlated 372 to wireline logs from wells in the subsurface formation. The geophysical wireline logs (e.g., DTCO, DTSM, and RHOB) are plotted 374 along with elastic properties (e.g., static Young's modulus, Estat) and petrophysical evaluations (e.g., PHIT) to determine a relationship between UCS and the wireline logs. The static Young's modulus can be determined based on wireline logs and petrophysical evaluations. The static Young's modulus can be calibrated with mechanical measurements from core samples (e.g., triaxial, multistage, or rebound hammer tests). Based on the correlations, the UCS log 362 is generated 376. Values form the laboratory measurements 364 can be used to validate the UCS log 362.

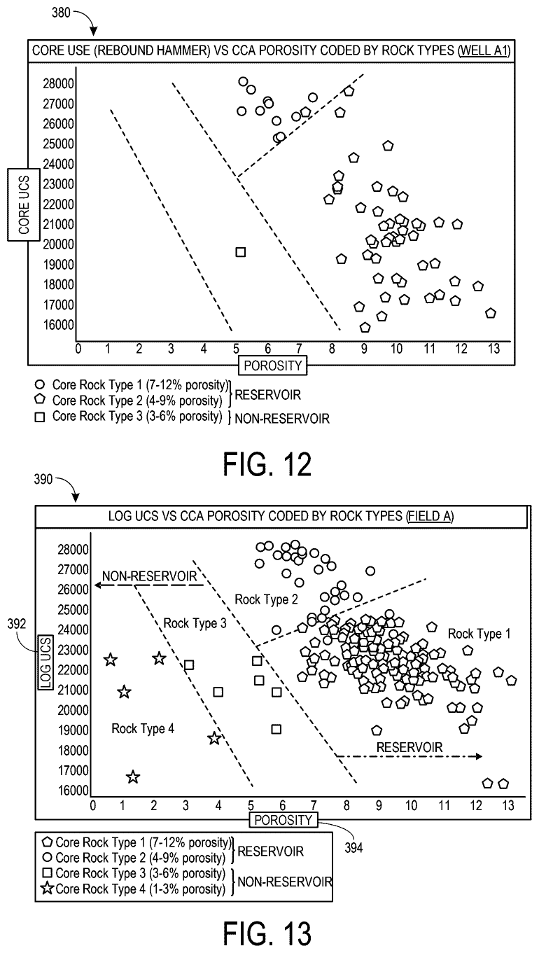

FIGS. 12 is a cross-plot 380 of core UCS 382 from micro-rebound hammer measurements on core samples from Well A1 in field A versus core porosity 384. The data points are coded by rock type providing segregation between the rock types within unique porosity ranges.

FIG. 13 is cross plot 390 of log UCS 392 versus core porosity 394 from multiple wells in field A. Using rock types provides good segregation of data for data from multiple wells. Rock types 1 and 2 indicate reservoir rock and rock types 3 and 4 indicate non-reservoir rock.

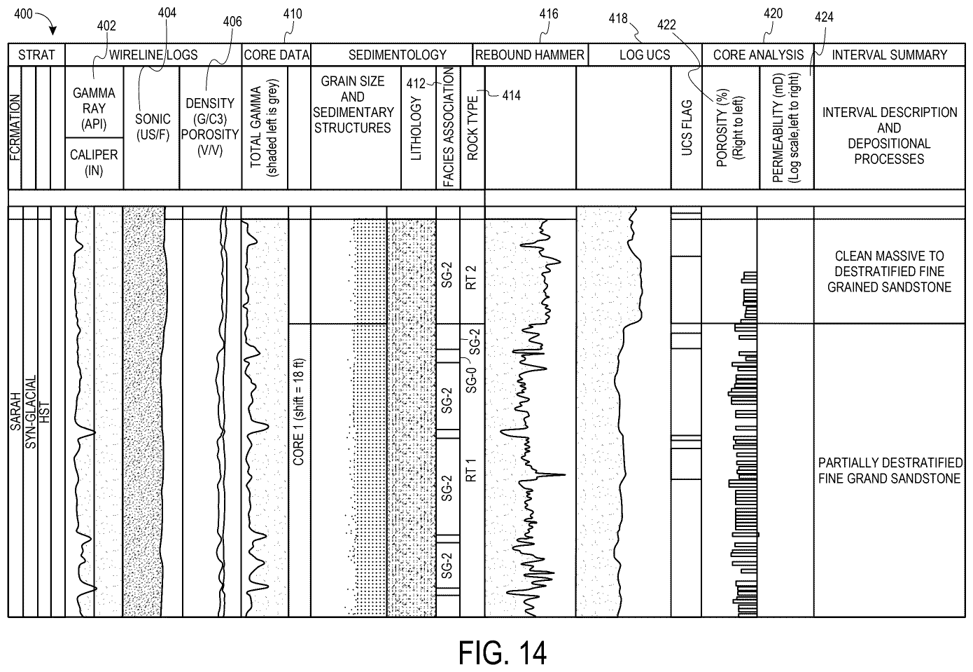

FIG. 14 is a composite plot 400 of conventional wireline logs (Gamma Ray 402, Sonic 404, Density and Neutron Porosity 408) along with the core description 410, core facies 412, core rock types 414, micro-rebound hammer core UCS 416, log UCS 418 and conventional core analysis 420 (porosity 422 and permeability 424) data for Well A1. Conventional wireline logs 402-406 cannot discriminate between the rock types 414 identified in core samples, which is a challenge for electrofacies propagation. However, both core UCS 416 and log UCS 420 do provide good discrimination.

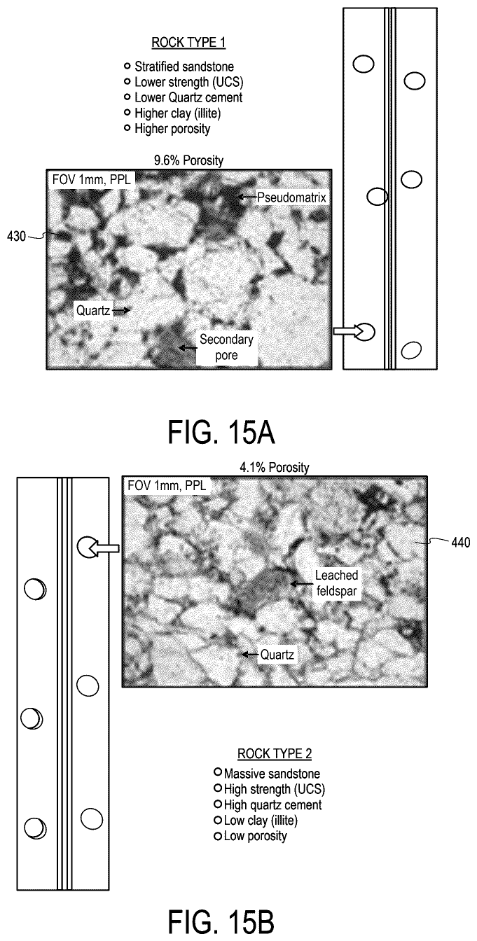

FIG. 15A illustrates the geological characteristics of RT1 in Well A1. RT1 is characterized by lower UCS seen in both core UCS 416 and log UCS 418 curves. The lower values record a softer rock than RT2 that is laminated/stratified in core depositional lithofacies as shown in the core photo 430. Laminations are caused by clay held within the rock which translates into a softer lithology. As demonstrated in FIGS. 9 and 10 the softness is caused by clay which also gives the rock its higher porosity (as microporosity). The clay held between the grains can be seen in the core photo 430. This slightly more clay rich lower UCS rock has higher conventional core analysis porosity values.

FIG. 15B illustrates the geological characteristics of RT2 in Well A1. RT2 is defined by higher UCS seen in both core UCS 416 and log UCS 418 curves. These higher values record a harder rock than RT1 that is generally massive (structureless) in core depositional lithofacies as seen in core photo 440. The structureless fabric is due to water escape from the rock soon after deposition, which led to removal of clays from the rock volume. As demonstrated in plot 344 in FIG. 10, there is an inverse correlation between clay and quartz cement. The lack of clay in the matrix resulted in appreciable quartz cementation, resulting in a harder rock with lower porosity. Extensive quartz cementation can be seen in the thin section photomicrograph (core photo 440) occluding porosity (e.g., low values seen in conventional core analysis data).

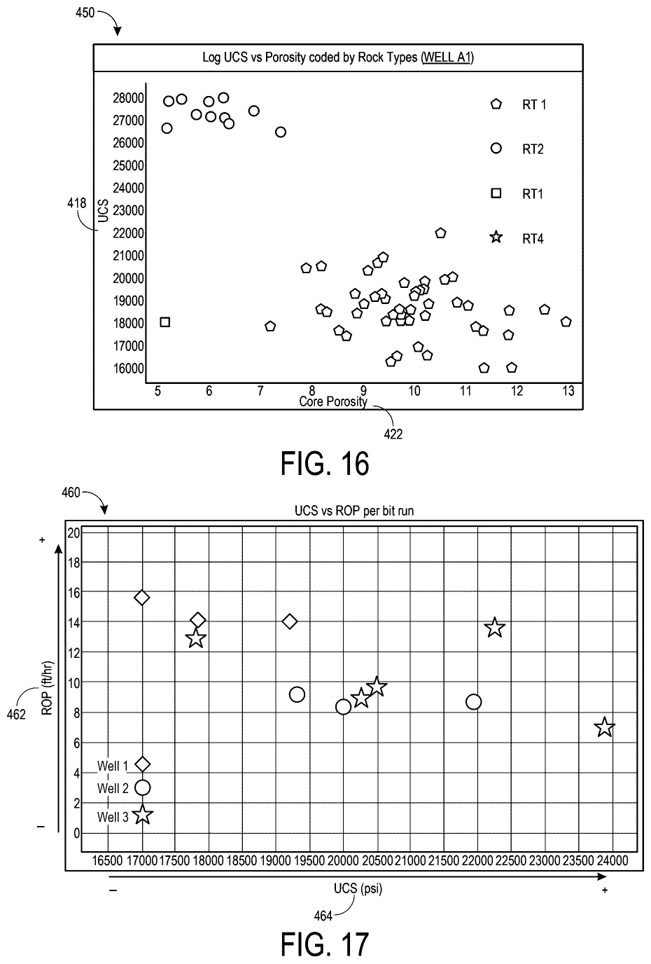

FIG. 16 is a cross plot 450 of log UCS 418 versus core porosity 422 coded by rock type 414. As also demonstrated in FIGS. 12-13, the log UCS 418 provides good discrimination between the rock types 414.

FIG. 17 is a cross plot 460 of ROP 462 versus UCS 464 from three wells in field A. As the UCS 464 increases, the ROP 462 decreases showing an inverse relationship between the two.

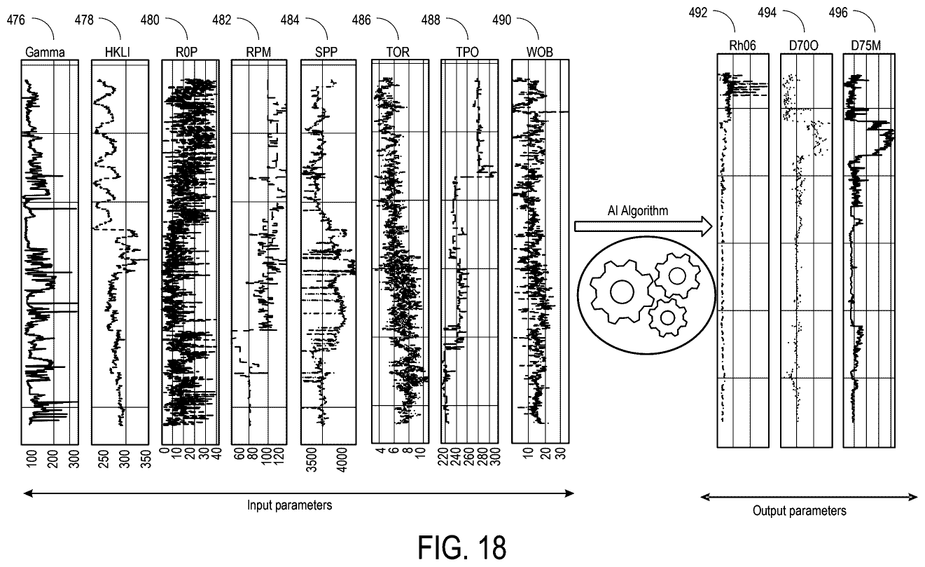

FIG. 18 is schematic showing example input parameters 470 to an artificial intelligence/machine learning model 472 to predict the drilling parameters 474. In this example, the input parameters 470 include a Gamma Ray log 476, a hook load log 478, an ROP log 480, an RPM log 482, a SPP log 484, a torque log 486, a flow rate log 488, and a WOB log 490. The output drilling parameters 474 include bulk density (RHOB) 492, compressional slowness (DTCO) 494, and shear slowness (DTSM) 496. In this example, several machine learning models are tested including random forest, XGBoost, and convolutional neural networks. The machine learning model that had the lowest RMSE value when each of the machine learning models is tested on a validation set can best capture the log response. The particular machine learning model may change based on the data in the training set.

FIG. 18 is a plot 500 of clusters formed by an unsupervised machine learning model. In this example, a K-Means clustering unsupervised machine learning models is used to separate the well log data into distinct clusters.

To reduce the dimensionality of the data, a Principal Component Analysis (PCA) was applied on gamma ray 502, RHOB 504, UCS 506, and PHIT 508. PCA is a dimensionality reduction technique that transforms the log measurements into new coordinates—principal components. The principal components represent the variance in the data, allowing for a more concise representation of information present in the wireline logs. In FIG. 18 the data is projected into two principal components PC1 510 and PC2 512 with explained variance of 90%. The projected data is then input into the K-Means model to separate the data into distinct clusters. K-Means clustering works by dividing the data into K determined clusters. Initially, a center point or centroid is picked to serve as each cluster center, and the distance between each point and centroid is calculated. The process is repeated until the averages do not change (or the change is below a threshold amount of change) or a predefined number of iterations is reached. The number of clusters (K) is predefined (e.g., by input from a user or determined by a data processing system).

FIG. 20 shows an elbow plot 520 that was constructed with various numbers of clusters 522 (K values) to aid in picking the predefined K value for plot 500. The within cluster sum of square (WCSS) 524 is plotted on the y-axis. The elbow 526 is indicated by a reduction in the decrease of WCSS 524 with increasing K 522. In elbow plot 520, the elbow 526 is determined to be at a K value of 4. To validate the clusters, the facies predicted from the core sample is compared to the clustered facies from the K-Means model.

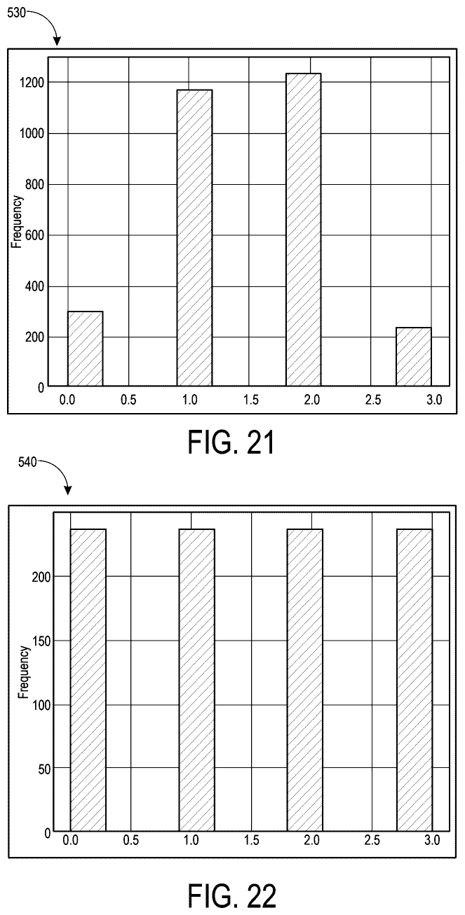

FIGS. 21-25 show plots related to determining rock types in field A using a supervised machine learning model that takes as input real time logging-while-drilling data. The logging-while-drilling data includes ROP logs, WOB logs, depth logs, gamma ray logs, and MSE logs. The ROP and WOB logs have been sanitized (e.g., cleaned or filtered) during conversion from time domain to depth domain. Training and predicting rock types occurs, for example, after the determination of the rock type clusters.

FIG. 21 shows an example bar chart 530 of the distribution of rock types within logging-while-drilling data. The distribution or rock types is uneven between the four rock types used in this example. RT1 and RT4 are underrepresented relative to RT2 and RT3. To correct for the uneven distribution, the RT2 and RT3 data points can be randomly under sampled.

FIG. 22 shows an example bar chart 540 where the distribution of data points among rock types has been equalized using random under sampling. In random under sampling, a data processing system randomly selects a particular number of data points from the identified groupings (e.g., RT2 and RT3). Equalizing the distribution of data points before training a supervised machine learning model helps to reduce bias in the trained model.

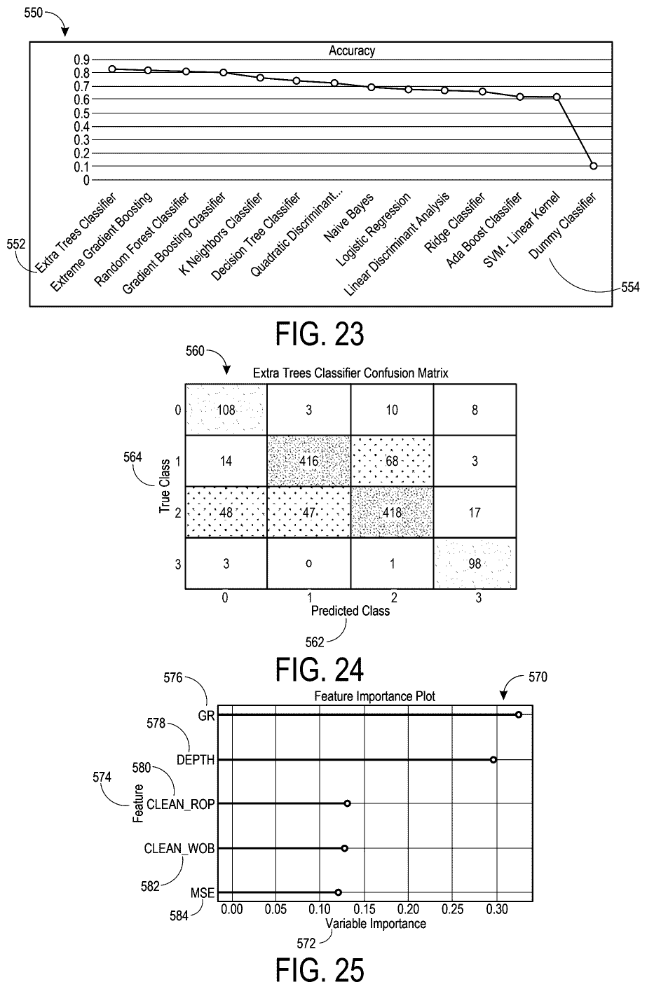

FIG. 23 is an accuracy plot 550 summarizing the prediction performance of several supervised machine learning models trained using the training dataset. In the accuracy plot 550, the extra trees classifier 552 performs the best. All of the supervised machine learning models performed substantially better than a dummy classifier 554. The accuracy can be determined based on the ratio of the number of true predictions for each class over the total number of predictions.

FIG. 24 is a confusion matrix 560 for the extra trees classifier 552. The confusion matrix 560 shows the predicted class 562 versus the true class 564 for each data sample. The extra trees classifier accurately classified 1040 samples out of a total of 1262 samples resulting in an overall accuracy of 82.4%.

FIG. 25 is a feature importance plot 570 for the extra trees classifier 552. The feature importance plot 570 shows the relative importance 572 of the input features 574. Here, the gamma ray 576 and depth 578 are the most important features. ROP 580, WOB 582, and MSE 584 are all nearly equally important but less than half of the importance of the gamma ray 576 and depth 578.

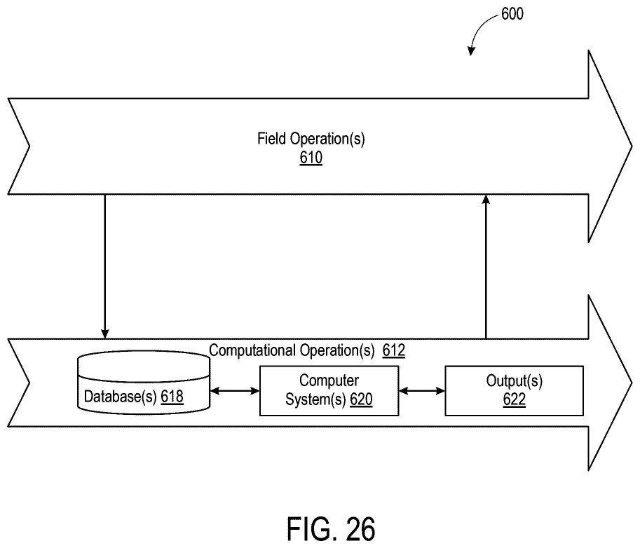

FIG. 26 illustrates hydrocarbon production operations 600 that include both one or more field operations 610 and one or more computational operations 612, which exchange information and control exploration for the production of hydrocarbons. In some implementations, outputs of techniques of the present disclosure (e.g., the method 300) can be performed before, during, or in combination with the hydrocarbon production operations 600, specifically, for example, either as field operations 610 or computational operations 612, or both.

Examples of field operations 610 include forming/drilling a wellbore, hydraulic fracturing, producing through the wellbore, injecting fluids (such as water) through the wellbore, to name a few. In some implementations, methods of the present disclosure can trigger or control the field operations 610. For example, the methods of the present disclosure can generate data from hardware/software including sensors and physical data gathering equipment (e.g., seismic sensors, well logging tools, flow meters, and temperature and pressure sensors). The methods of the present disclosure can include transmitting the data from the hardware/software to the field operations 610 and responsively triggering the field operations 610 including, for example, generating plans and signals that provide feedback to and control physical components of the field operations 610. Alternatively, or in addition, the field operations 610 can trigger the methods of the present disclosure. For example, implementing physical components (including, for example, hardware, such as sensors) deployed in the field operations 610 can generate plans and signals that can be provided as input or feedback (or both) to the methods of the present disclosure.

Examples of computational operations 612 include one or more computer systems 620 that include one or more processors and computer-readable media (e.g., non-transitory computer-readable media) operatively coupled to the one or more processors to execute computer operations to perform the methods of the present disclosure. The computational operations 612 can be implemented using one or more databases 618, which store data received from the field operations 610 and/or generated internally within the computational operations 612 (e.g., by implementing the methods of the present disclosure) or both. For example, the one or more computer systems 620 process inputs from the field operations 610 to assess conditions in the physical world, the outputs of which are stored in the databases 618. For example, seismic sensors of the field operations 610 can be used to perform a seismic survey to map subterranean features, such as facies and faults. In performing a seismic survey, seismic sources (e.g., seismic vibrators or explosions) generate seismic waves that propagate in the earth and seismic receivers (e.g., geophones) measure reflections generated as the seismic waves interact with boundaries between layers of a subsurface formation. The source and received signals are provided to the computational operations 612 where they are stored in the databases 618 and analyzed by the one or more computer systems 620.

In some implementations, one or more outputs 622 generated by the one or more computer systems 620 can be provided as feedback/input to the field operations 610 (either as direct input or stored in the databases 618). The field operations 610 can use the feedback/input to control physical components used to perform the field operations 610 in the real world.

For example, the computational operations 612 can process the seismic data to generate three-dimensional (3D) maps of the subsurface formation. The computational operations 612 can use these 3D maps to provide plans for locating and drilling exploratory wells. In some operations, the exploratory wells are drilled using logging-while-drilling (LWD) techniques which incorporate logging tools into the drill string. LWD techniques can enable the computational operations 612 to process new information about the formation and control the drilling to adjust to the observed conditions in real-time.

The one or more computer systems 620 can update the 3D maps of the subsurface formation as information from one exploration well is received and the computational operations 612 can adjust the location of the next exploration well based on the updated 3D maps. Similarly, the data received from production operations can be used by the computational operations 612 to control components of the production operations. For example, production well and pipeline data can be analyzed to predict slugging in pipelines leading to a refinery and the computational operations 612 can control machine operated valves upstream of the refinery to reduce the likelihood of plant disruptions that run the risk of taking the plant offline.

In some implementations of the computational operations 612, customized user interfaces can present intermediate or final results of the above-described processes to a user. Information can be presented in one or more textual, tabular, or graphical formats, such as through a dashboard. The information can be presented at one or more on-site locations (such as at an oil well or other facility), on the Internet (such as on a webpage), on a mobile application (or app), or at a central processing facility.

The presented information can include feedback, such as changes in parameters or processing inputs, that the user can select to improve a production environment, such as in the exploration, production, and/or testing of petrochemical processes or facilities. For example, the feedback can include parameters that, when selected by the user, can cause a change to, or an improvement in, drilling parameters (including drill bit speed and direction) or overall production of a gas or oil well. The feedback, when implemented by the user, can improve the speed and accuracy of calculations, streamline processes, improve models, and solve problems related to efficiency, performance, safety, reliability, costs, downtime, and the need for human interaction.

In some implementations, the feedback can be implemented in real-time, such as to provide an immediate or near-immediate change in operations or in a model. The term real-time (or similar terms as understood by one of ordinary skill in the art) means that an action and a response are temporally proximate such that an individual perceives the action and the response occurring substantially simultaneously. For example, the time difference for a response to display (or for an initiation of a display) of data following the individual's action to access the data can be less than 1 millisecond (ms), less than 1 second(s), or less than 5 s. While the requested data need not be displayed (or initiated for display) instantaneously, it is displayed (or initiated for display) without any intentional delay, taking into account processing limitations of a described computing system and time required to, for example, gather, accurately measure, analyze, process, store, or transmit the data.

Events can include readings or measurements captured by downhole equipment such as sensors, pumps, bottom hole assemblies, or other equipment. The readings or measurements can be analyzed at the surface, such as by using applications that can include modeling applications and machine learning. The analysis can be used to generate changes to settings of downhole equipment, such as drilling equipment. In some implementations, values of parameters or other variables that are determined can be used automatically (such as through using rules) to implement changes in oil or gas well exploration, production/drilling, or testing. For example, outputs of the present disclosure can be used as inputs to other equipment and/or systems at a facility. This can be especially useful for systems or various pieces of equipment that are located several meters or several miles apart or are located in different countries or other jurisdictions.

FIG. 27 is a block diagram of an example computer system 700 used to provide computational functionalities associated with described algorithms, methods, functions, processes, flows, and procedures described in the present disclosure, according to some implementations of the present disclosure. The illustrated computer 702 is intended to encompass any computing device such as a server, a desktop computer, a laptop/notebook computer, a wireless data port, a smart phone, a personal data assistant (PDA), a tablet computing device, or one or more processors within these devices, including physical instances, virtual instances, or both. The computer 702 can include input devices such as keypads, keyboards, and touch screens that can accept user information. Also, the computer 702 can include output devices that can convey information associated with the operation of the computer 702. The information can include digital data, visual data, audio information, or a combination of information. The information can be presented in a graphical user interface (UI) (or GUI).

The computer 702 can serve in a role as a client, a network component, a server, a database, a persistency, or components of a computer system for performing the subject matter described in the present disclosure. The illustrated computer 702 is communicably coupled with a network 730. In some implementations, one or more components of the computer 702 can be configured to operate within different environments, including cloud-computing-based environments, local environments, global environments, and combinations of environments.

At a high level, the computer 702 is an electronic computing device operable to receive, transmit, process, store, and manage data and information associated with the described subject matter. According to some implementations, the computer 702 can also include, or be communicably coupled with, an application server, an email server, a web server, a caching server, a streaming data server, or a combination of servers.

The computer 702 can receive requests over network 730 from a client application (for example, executing on another computer 702). The computer 702 can respond to the received requests by processing the received requests using software applications. Requests can also be sent to the computer 702 from internal users (for example, from a command console), external (or third) parties, automated applications, entities, individuals, systems, and computers.

Each of the components of the computer 702 can communicate using a system bus 703. In some implementations, any or all of the components of the computer 702, including hardware or software components, can interface with each other or the interface 704 (or a combination of both), over the system bus 703. Interfaces can use an application programming interface (API) 712, a service layer 713, or a combination of the API 712 and service layer 713. The API 712 can include specifications for routines, data structures, and object classes. The API 712 can be either computer-language independent or dependent. The API 712 can refer to a complete interface, a single function, or a set of APIs.

The service layer 713 can provide software services to the computer 702 and other components (whether illustrated or not) that are communicably coupled to the computer 702. The functionality of the computer 702 can be accessible for all service consumers using this service layer. Software services, such as those provided by the service layer 713, can provide reusable, defined functionalities through a defined interface. For example, the interface can be software written in JAVA, C++, or a language providing data in extensible markup language (XML) format. While illustrated as an integrated component of the computer 702, in alternative implementations, the API 712 or the service layer 713 can be stand-alone components in relation to other components of the computer 702 and other components communicably coupled to the computer 702. Moreover, any or all parts of the API 712 or the service layer 713 can be implemented as child or sub-modules of another software module, enterprise application, or hardware module without departing from the scope of the present disclosure.

The computer 702 includes an interface 704. Although illustrated as a single interface 704 in FIG. 7, two or more interfaces 704 can be used according to particular needs, desires, or particular implementations of the computer 702 and the described functionality. The interface 704 can be used by the computer 702 for communicating with other systems that are connected to the network 730 (whether illustrated or not) in a distributed environment. Generally, the interface 704 can include, or be implemented using, logic encoded in software or hardware (or a combination of software and hardware) operable to communicate with the network 730. More specifically, the interface 704 can include software supporting one or more communication protocols associated with communications. As such, the network 730 or the interface's hardware can be operable to communicate physical signals within and outside of the illustrated computer 702.

The computer 702 includes a processor 705. Although illustrated as a single processor 705 in FIG. 7, two or more processors 705 can be used according to particular needs, desires, or particular implementations of the computer 702 and the described functionality. Generally, the processor 705 can execute instructions and can manipulate data to perform the operations of the computer 702, including operations using algorithms, methods, functions, processes, flows, and procedures as described in the present disclosure.

The computer 702 also includes a database 706 that can hold data for the computer 702 and other components connected to the network 730 (whether illustrated or not). For example, database 706 can be an in-memory, conventional, or a database storing data consistent with the present disclosure. In some implementations, database 706 can be a combination of two or more different database types (for example, hybrid in-memory and conventional databases) according to particular needs, desires, or particular implementations of the computer 702 and the described functionality. Although illustrated as a single database 706 in FIG. 7, two or more databases (of the same, different, or combination of types) can be used according to particular needs, desires, or particular implementations of the computer 702 and the described functionality. While database 706 is illustrated as an internal component of the computer 702, in alternative implementations, database 706 can be external to the computer 702.

The computer 702 also includes a memory 707 that can hold data for the computer 702 or a combination of components connected to the network 730 (whether illustrated or not). Memory 707 can store any data consistent with the present disclosure. In some implementations, memory 707 can be a combination of two or more different types of memory (for example, a combination of semiconductor and magnetic storage) according to particular needs, desires, or particular implementations of the computer 702 and the described functionality. Although illustrated as a single memory 707 in FIG. 7, two or more memories 707 (of the same, different, or combination of types) can be used according to particular needs, desires, or particular implementations of the computer 702 and the described functionality. While memory 707 is illustrated as an internal component of the computer 702, in alternative implementations, memory 707 can be external to the computer 702.

The application 708 can be an algorithmic software engine providing functionality according to particular needs, desires, or particular implementations of the computer 702 and the described functionality. For example, application 708 can serve as one or more components, modules, or applications. Further, although illustrated as a single application 708, the application 708 can be implemented as multiple applications 708 on the computer 702. In addition, although illustrated as internal to the computer 702, in alternative implementations, the application 708 can be external to the computer 702.

The computer 702 can also include a power supply 714. The power supply 714 can include a rechargeable or non-rechargeable battery that can be configured to be either user- or non-user-replaceable. In some implementations, the power supply 714 can include power-conversion and management circuits, including recharging, standby, and power management functionalities. In some implementations, the power-supply 714 can include a power plug to allow the computer 702 to be plugged into a wall socket or a power source to, for example, power the computer 702 or recharge a rechargeable battery.

There can be any number of computers 702 associated with, or external to, a computer system containing computer 702, with each computer 702 communicating over network 730. Further, the terms “client,” “user,” and other appropriate terminology can be used interchangeably, as appropriate, without departing from the scope of the present disclosure. Moreover, the present disclosure contemplates that many users can use one computer 702 and one user can use multiple computers 702.

Implementations of the subject matter and the functional operations described in this specification can be implemented in digital electronic circuitry, in tangibly embodied computer software or firmware, in computer hardware, including the structures disclosed in this specification and their structural equivalents, or in combinations of one or more of them. Software implementations of the described subject matter can be implemented as one or more computer programs. Each computer program can include one or more modules of computer program instructions encoded on a tangible, non transitory, computer-readable computer-storage medium for execution by, or to control the operation of, data processing apparatus. Alternatively, or additionally, the program instructions can be encoded in/on an artificially generated propagated signal. The example, the signal can be a machine-generated electrical, optical, or electromagnetic signal that is generated to encode information for transmission to suitable receiver apparatus for execution by a data processing apparatus. The computer-storage medium can be a machine-readable storage device, a machine-readable storage substrate, a random or serial access memory device, or a combination of computer-storage mediums.

The terms “data processing apparatus,” “computer,” and “electronic computer device” (or equivalent as understood by one of ordinary skill in the art) refer to data processing hardware. For example, a data processing apparatus can encompass all kinds of apparatus, devices, and machines for processing data, including by way of example, a programmable processor, a computer, or multiple processors or computers. The apparatus can also include special purpose logic circuitry including, for example, a central processing unit (CPU), a field programmable gate array (FPGA), or an application specific integrated circuit (ASIC). In some implementations, the data processing apparatus or special purpose logic circuitry (or a combination of the data processing apparatus or special purpose logic circuitry) can be hardware- or software-based (or a combination of both hardware- and software-based). The apparatus can optionally include code that creates an execution environment for computer programs, for example, code that constitutes processor firmware, a protocol stack, a database management system, an operating system, or a combination of execution environments. The present disclosure contemplates the use of data processing apparatuses with or without conventional operating systems, for example LINUX, UNIX, WINDOWS, MAC OS, ANDROID, or IOS.

The methods, processes, or logic flows described in this specification can be performed by one or more programmable computers executing one or more computer programs to perform functions by operating on input data and generating output. The methods, processes, or logic flows can also be performed by, and apparatus can also be implemented as, special purpose logic circuitry, for example, a CPU, an FPGA, or an ASIC.

Computer readable media (transitory or non-transitory, as appropriate) suitable for storing computer program instructions and data can include all forms of permanent/non-permanent and volatile/non-volatile memory, media, and memory devices. Computer readable media can include, for example, semiconductor memory devices such as random access memory (RAM), read only memory (ROM), phase change memory (PRAM), static random access memory (SRAM), dynamic random access memory (DRAM), erasable programmable read-only memory (EPROM), electrically erasable programmable read-only memory (EEPROM), and flash memory devices. Computer readable media can also include, for example, magnetic devices such as tape, cartridges, cassettes, and internal/removable disks.

While this specification contains many specific implementation details, these should not be construed as limitations on the scope of what may be claimed, but rather as descriptions of features that may be specific to particular implementations. Certain features that are described in this specification in the context of separate implementations can also be implemented, in combination, in a single implementation. Conversely, various features that are described in the context of a single implementation can also be implemented in multiple implementations, separately, or in any suitable sub-combination. Moreover, although previously described features may be described as acting in certain combinations and even initially claimed as such, one or more features from a claimed combination can, in some cases, be excised from the combination, and the claimed combination may be directed to a sub-combination or variation of a sub-combination.

Particular implementations of the subject matter have been described. Other implementations, alterations, and permutations of the described implementations are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations may be considered optional), to achieve desirable results. In certain circumstances, multitasking or parallel processing (or a combination of multitasking and parallel processing) may be advantageous and performed as deemed appropriate.

Moreover, the separation or integration of various system modules and components in the previously described implementations should not be understood as requiring such separation or integration in all implementations, and it should be understood that the described program components and systems can generally be integrated together in a single software product or packaged into multiple software products.

Accordingly, the previously described example implementations do not define or constrain the present disclosure. Other changes, substitutions, and alterations are also possible without departing from the spirit and scope of the present disclosure.

Furthermore, any claimed implementation is considered to be applicable to at least a computer-implemented method; a non-transitory, computer-readable medium storing computer-readable instructions to perform the computer-implemented method; and a computer system comprising a computer memory interoperably coupled with a hardware processor configured to perform the computer-implemented method or the instructions stored on the non-transitory, computer-readable medium.

A number of embodiments of these systems and methods have been described. Nevertheless, it will be understood that various modifications may be made without departing from the spirit and scope of this disclosure. Accordingly, other embodiments are within the scope of the following claims.

### EXAMPLES

In an example implementation, a method includes obtaining well log data and core sample data of a subsurface formation; generating, based on the well log data and the core sample data, an unconfined compressive strength log for the subsurface formation; using an unsupervised machine learning model to form rock type clusters based on the unconfined compressive strength log and the well log data; forming a training dataset including the well log data, the training dataset labeled based on the rock type clusters; training a supervised machine learning model using the training dataset; while drilling a well in the subsurface formation, obtaining logging-while-drilling data from drilling equipment used to drill the well; and determining rock types in the subsurface formation using the supervised machine learning model and the logging-while-drilling data.

An aspect combinable with the example implementation includes in response to determining the rock types, controlling the drilling equipment based on the determined rock types.

In another aspect combinable with any of the previous aspects, controlling the drilling equipment includes controlling a rate of penetration of the drilling equipment or steering the drilling equipment.

In another aspect combinable with any of the previous aspects, forming rock type clusters includes applying principal component analysis to the well log data and the unconfined compressive strength log to reduce inputs to the unsupervised machine learning model.

In another aspect combinable with any of the previous aspects, the well log data includes a total porosity log, a density log, and a gamma ray log, and wherein the unsupervised machine learning model takes as input two principal components identified by the principal component analysis.

In another aspect combinable with any of the previous aspects, the well log data and the logging-while-drilling data include one or more of a rate of penetration log, a gamma ray log, a weight on bit log, and a mechanical specific energy log.

In another aspect combinable with any of the previous aspects, generating the unconfined compressive strength log includes generating additional log data using a machine learning model that takes as input the well log data and outputs the additional log data, wherein the well log data includes rate of penetration data and drilling parameters.

In another aspect combinable with any of the previous aspects, generating the unconfined compressive strength log data includes validating the unconfined compressive strength log data with one or more of micro-rebound hammer uniaxial compressive strength data, thin section point count data, and x-ray diffraction mineralogical data.

Another aspect combinable with any of the previous aspects includes updating a three-dimensional static and dynamic reservoir model based on the determined rock types.

In another aspect combinable with any of the previous aspects, obtaining the drilling while logging data includes obtaining time-domain drilling while logging data, and converting the time-domain drilling while logging data to depth domain drilling while logging data for input to the supervised machine learning model.

In another example implementation, a system includes at least one processor and a memory storing instructions that, when executed by the at least one processor, cause the at least one processor to perform operations including obtaining well log data and core sample data of a subsurface formation; generating, based on the well log data and the core sample data, an unconfined compressive strength log for the subsurface formation; using an unsupervised machine learning model to form rock type clusters based on the unconfined compressive strength log and the well log data; forming a training dataset including the well log data, the training dataset labeled based on the rock type clusters; training a supervised machine learning model using the training dataset; while drilling a well in the subsurface formation, obtaining logging-while-drilling data from drilling equipment used to drill the well; and determining rock types in the subsurface formation using the supervised machine learning model and the logging-while-drilling data.

In an aspect combinable with the example implementation, the operations include in response to determining the rock types, controlling a rate of penetration of the drilling equipment or steering the drilling equipment.

In another aspect combinable with any of the previous aspects, forming rock type clusters includes applying principal component analysis to the well log data and the unconfined compressive strength log to reduce inputs to the unsupervised machine learning model.

In another aspect combinable with any of the previous aspects, generating the unconfined compressive strength log includes generating additional log data using a machine learning model that takes as input the well log data and outputs the additional log data, wherein the well log data comprises rate of penetration data and drilling parameters; and validating the unconfined compressive strength log data with one or more of micro-rebound hammer uniaxial compressive strength data, thin section point count data, and x-ray diffraction mineralogical data.

In another aspect combinable with any of the previous aspects, the operations include updating a three-dimensional static and dynamic reservoir model based on the determined rock types.

In another aspect combinable with any of the previous aspects, obtaining the drilling while logging data includes obtaining time-domain drilling while logging data; and operations include converting the time-domain drilling while logging data to depth domain drilling while logging data for input to the supervised machine learning model, where the well log data and the logging-while-drilling data include one or more of a rate of penetration log, a gamma ray log, a weight on bit log, and a mechanical specific energy log.

In another example implementation, one or more non-transitory machine-readable storage devices storing instructions, the instructions being executable by one or more processors, to cause performance of operations include obtaining well log data and core sample data of a subsurface formation; generating, based on the well log data and the core sample data, an unconfined compressive strength log for the subsurface formation; using an unsupervised machine learning model to form rock type clusters based on the unconfined compressive strength log and the well log data; forming a training dataset including the well log data, the training dataset labeled based on the rock type clusters; training a supervised machine learning model using the training dataset; while drilling a well in the subsurface formation, obtaining logging-while-drilling data from drilling equipment used to drill the well; and determining rock types in the subsurface formation using the supervised machine learning model and the logging-while-drilling data.

In an aspect combinable with the example implementation, the operations include in response to determining the rock types, controlling a rate of penetration of the drilling equipment, steering the drilling equipment, or updating a three-dimensional static and dynamic reservoir model based on the determined rock types.

In another aspect combinable with any of the previous aspects, generating the unconfined compressive strength log includes generating additional log data using a machine learning model that takes as input the well log data and outputs the additional log data, where the well log data includes rate of penetration data and drilling parameters; and validating the unconfined compressive strength log data with one or more of micro-rebound hammer uniaxial compressive strength data, thin section point count data, and x-ray diffraction mineralogical data.

In another aspect combinable with any of the previous aspects, obtaining the drilling while logging data includes obtaining time-domain drilling while logging data, and the operations include converting the time-domain drilling while logging data to depth domain drilling while logging data for input to the supervised machine learning model, where the well log data and the logging-while-drilling data include one or more of a rate of penetration log, a gamma ray log, a weight on bit log, and a mechanical specific energy log.

What is claimed is:

1. A method comprising:
obtaining well log data and core sample data of a subsurface formation;
generating, based on the well log data and the core sample data, an unconfined compressive strength log for the subsurface formation;
using an unsupervised machine learning model to form rock type clusters based on the unconfined compressive strength log and the well log data;
forming a training dataset including the well log data, the training dataset labeled based on the rock type clusters;
training a supervised machine learning model using the training dataset;
while drilling a well in the subsurface formation, obtaining logging-while-drilling data from drilling equipment used to drill the well; and
determining rock types in the subsurface formation using the supervised machine learning model and the logging-while-drilling data.

2. The method of claim 1, further comprising in response to determining the rock types, controlling the drilling equipment based on the determined rock types.

3. The method of claim 2, wherein controlling the drilling equipment comprises controlling a rate of penetration of the drilling equipment or steering the drilling equipment.

4. The method of claim 1, wherein forming rock type clusters comprises applying principal component analysis to the well log data and the unconfined compressive strength log to reduce inputs to the unsupervised machine learning model.

5. The method of claim 4, wherein the well log data comprises a total porosity log, a density log, and a gamma ray log, and wherein the unsupervised machine learning model takes as input two principal components identified by the principal component analysis.

6. The method of claim 1, wherein the well log data and the logging-while-drilling data comprise one or more of a rate of penetration log, a gamma ray log, a weight on bit log, and a mechanical specific energy log.

7. The method of claim 1, wherein generating the unconfined compressive strength log comprises generating additional log data using a machine learning model that takes as input the well log data and outputs the additional log data, wherein the well log data comprises rate of penetration data and drilling parameters.

8. The method of claim 1, wherein generating the unconfined compressive strength log data comprises validating the unconfined compressive strength log data with one or more of micro-rebound hammer uniaxial compressive strength data, thin section point count data, and x-ray diffraction mineralogical data.

9. The method of claim 1, further comprising updating a three-dimensional static and dynamic reservoir model based on the determined rock types.

10. The method of claim 1, wherein obtaining the drilling while logging data comprises obtaining time-domain drilling while logging data, and
wherein the method further comprises converting the time-domain drilling while logging data to depth domain drilling while logging data for input to the supervised machine learning model.

11. A system comprising:
at least one processor and a memory storing instructions that, when executed by the at least one processor, cause the at least one processor to perform operations comprising:
obtaining well log data and core sample data of a subsurface formation;
generating, based on the well log data and the core sample data, an unconfined compressive strength log for the subsurface formation;
using an unsupervised machine learning model to form rock type clusters based on the unconfined compressive strength log and the well log data;
forming a training dataset including the well log data, the training dataset labeled based on the rock type clusters;
training a supervised machine learning model using the training dataset;
while drilling a well in the subsurface formation, obtaining logging-while-drilling data from drilling equipment used to drill the well; and
determining rock types in the subsurface formation using the supervised machine learning model and the logging-while-drilling data.

12. The system of claim 11, wherein the operations further comprise in response to determining the rock types, controlling a rate of penetration of the drilling equipment or steering the drilling equipment.

13. The system of claim 11, wherein forming rock type clusters comprises applying principal component analysis to the well log data and the unconfined compressive strength log to reduce inputs to the unsupervised machine learning model.

14. The system of claim 11, wherein generating the unconfined compressive strength log comprises:
generating additional log data using a machine learning model that takes as input the well log data and outputs the additional log data, wherein the well log data comprises rate of penetration data and drilling parameters; and
validating the unconfined compressive strength log data with one or more of micro-rebound hammer uniaxial compressive strength data, thin section point count data, and x-ray diffraction mineralogical data.

15. The system of claim 11, wherein the operations further comprise updating a three-dimensional static and dynamic reservoir model based on the determined rock types.

16. The system of claim 11, wherein obtaining the drilling while logging data comprises obtaining time-domain drilling while logging data; and
wherein the operations further comprise converting the time-domain drilling while logging data to depth domain drilling while logging data for input to the supervised machine learning model,
wherein the well log data and the logging-while-drilling data comprise one or more of a rate of penetration log, a gamma ray log, a weight on bit log, and a mechanical specific energy log.

17. One or more non-transitory machine-readable storage devices storing instructions, the instructions being executable by one or more processors, to cause performance of operations comprising:
obtaining well log data and core sample data of a subsurface formation;
generating, based on the well log data and the core sample data, an unconfined compressive strength log for the subsurface formation;
using an unsupervised machine learning model to form rock type clusters based on the unconfined compressive strength log and the well log data;
forming a training dataset including the well log data, the training dataset labeled based on the rock type clusters;
training a supervised machine learning model using the training dataset;
while drilling a well in the subsurface formation, obtaining logging-while-drilling data from drilling equipment used to drill the well; and
determining rock types in the subsurface formation using the supervised machine learning model and the logging-while-drilling data.

18. The one or more non-transitory machine readable storage devices of claim 17, wherein the operations further comprise:
in response to determining the rock types, controlling a rate of penetration of the drilling equipment, steering the drilling equipment, or updating a three-dimensional static and dynamic reservoir model based on the determined rock types.

19. The one or more non-transitory machine readable storage devices of claim 17, wherein generating the unconfined compressive strength log comprises:
generating additional log data using a machine learning model that takes as input the well log data and outputs the additional log data, wherein the well log data comprises rate of penetration data and drilling parameters; and
validating the unconfined compressive strength log data with one or more of micro-rebound hammer uniaxial compressive strength data, thin section point count data, and x-ray diffraction mineralogical data.

20. The one or more non-transitory machine readable storage devices of claim 17, wherein obtaining the drilling while logging data comprises obtaining time-domain drilling while logging data, and
wherein the operations further comprise converting the time-domain drilling while logging data to depth domain drilling while logging data for input to the supervised machine learning model, wherein the well log data and the logging-while-drilling data comprise one or more of a rate of penetration log, a gamma ray log, a weight on bit log, and a mechanical specific energy log.
